(19) United States
(12) Patent Application Publication
(10) Pub. No.: US 2009/0117536 A1
(43) Pub. Date: May 7, 2009

(54) METHOD AND DEVICE FOR BACTERIAL SAMPLING

(75) Inventors: Michael Mattey, Hertfordshire (GB); Robert Ivan Wilkinson, Hertfordshire (GB)

Correspondence Address: THOMPSON HINE L.L.P.;Intellectual Property Group, P.O. BOX 8801, DAYTON, OH, 45401-8801, US

(73) Assignee: Blaze Venture Technologies Limited, Hertfordshire (GB)

(21) Appl. No.: 11/817,494

(22) PCT Filed: Mar. 6, 2006
(86) PCT No.: PCT/GB2006/000790

§ 371 Date: May 19, 2008

(30) Foreign Application Priority Data
Mar. 4, 2005 (GB) 0504537.2
Mar. 18, 2005 (GB) 0505617.1

(51) Int. Cl. C12Q 1/70; C12M 1/34

(52) U.S. Cl. 435/5; 435/288.7

(57) ABSTRACT

A bacterial detection sampling device comprising: a sampling medium for receiving a bacterial sample; and a plurality of bacteriophage. The bacteriophage are located on or in the sampling medium. Each bacteriophage comprises a nucleic acid encoding a protein capable of emitting light at an output wavelength.

This invention relates to techniques for taking samples, and determining the presence of bacteria therein. It is primarily directed at taking samples from surfaces, as part of the process of maintaining a clean and hygienic environment in indoor premises. The invention has especial application in hospitals and like establishments. The invention also relates to products that can be used in such techniques.

Methicillin resistant Staphylococcus Aureus (MRSA) is a variety of bacteria that is resistant to most modern antibiotics. MRSA organisms can generally be tolerated by healthy individuals, but if they pass to someone who is already unwell, then this may lead to more serious infection. As a consequence, such organisms can be carried by healthy individuals without causing any problem, but in a hospital or other environment where more vulnerable people may be located, there is a serious risk of infection. MRSA is carried on and can remain on skin and other surfaces for long periods, and readily transfer from surface to surface. As a consequence, it can be carried directly and indirectly between individuals, and to individuals likely to be infected.

Current techniques for detecting MRSA are somewhat laborious, requiring a laboratory culture process and typically taking two to three days. Within such a time span, any MRSA that was present can have become widely spread in the respective environment.

In one aspect, the present invention is directed at a sampling technique which can dramatically reduce time for detecting the presence of bacteria, and particularly but not exclusively, MRSA in either its growing or its dormant form. It avoids the use of complex laboratory equipments, and does not require the services of a qualified microbiologist. The techniques of the present invention use bacteriophage that seek out and attach to a target bacteria. The bacteriophage used comprises a nucleic acid encoding a fluorescing protein, which protein is responsive to light of a first wavelength by emitting light of a second wavelength. When the bacteriophage contacts the target bacteria, the bacteriophage multiplies. As a consequence, when the multiplied bacteriophage is exposed to light of the first wavelength, the quantity of light emitted at the second wavelength is increased. This enables the ready detection of the presence of the bacteria by optical processes. In practice the optical process can be controlled such that it is normally necessary only to detect the emission of light at the second wavelength to establish whether the target bacteria is present.

In practicing the invention, the preferred protein in the bacteriophage is Green fluorescent protein (GFP) which, when exposed to light of wavelength 395 nm, emits light of wavelength 510 nm. Whichever protein is used, the bacteriophage can be selected to be specific for one strain of bacteria, and can be specific to a strain of MRSA. The particularly virulent strains of MRSA with which the present invention has a particular but not exclusive concern, are strains 3, 15 and 16.

In practicing the method of the invention, the selected bacteriophage can be disposed on a solid substrate, and are preferably immobilised on the substrate. Immobilisation may be accomplished by creating a covalent bond, typically supplemented by a coupling agent. The immobilisation and stabilisation of viruses including bacteriophage, to solid substrates is discussed in International Patent Publication No: WO 03/093462, in the name of The University of Strathclyde. The bacteriophage is preferably immobilised via its head group, leaving the tail group free.

Particularly preferred substrates for bacterial detection sampling devices according to the invention are:
1) nylon or another polymer with amino or carboxy surface groups and the coupling agent is carbodiimide or glutaraldehyde;
2) cellulose or another hydroxyl-containing polymer and the coupling agent comprises vinylsulfonylethylene ether or triazine;
3) polythene or similar polymer and the coupling agent comprises corona discharge or permanganate oxidation.

In the practice of the invention, a typical sample suspected of carrying bacteria will be a surface. Such a surface can be wiped with a substrate carrying the bacteriophage, with the substrate (or the surface) then being exposed to light of the first wavelength to determine the presence of the bacteria. However, if the suspect sample is a liquid, then the bacteriophage either alone or on a substrate, can be immersed in the liquid, which is then exposed to light as aforesaid. There may be some benefit in any event in immersing the bacterial sample in a liquid after contact with the bacteriophage to enable the bacteria to grow. A suitable liquid medium for this purpose would be an aqueous nutrient medium containing a carbon source such as glucose.

It will be appreciated that the actual detecting step in practicing the present invention can use relatively straightforward optical techniques. The fluorescence or increased fluorescence of the bacteriophage can be observed using a photodiode or photomultiplier tube for example, and there is no necessity to quantify the level of fluorescence other than relative to that of the bacteriophage prior to multiplication upon contact with the bacteria. As this initial level of fluorescence can be relatively low, it will be apparent that the identification of substantial fluorescence should be sufficient to establish whether the targeted bacteria is present.

The sampling device itself can also be a quite straightforward unit. Typically it will comprise a holder upon which a substrate is mounted for bearing the bacteriophage. The substrate can be in the form of a pad or swab, and its mounting may be facilitated by selected an appropriate configuration such as a disc or annulus. It could bear an adhesive layer for attachment to an instrument for presenting it to the sample surface or environment suspected of bearing the targeted bacteria.

According to another aspect of the present invention, there is provided a means of facilitating the sampling techniques described above. It comprises a test element, such as a card, on which is located the selected bacteriophage. The sample suspected of carrying the target bacteria is wiped with a swab, which is in turn wiped on the element to engage any bacteria picked up from the sample with the bacteriophage. The element is then introduced into a sensor unit, which will establish whether the fluorescence caused by the bacteriophage has increased as a result of contact with bacteria. The unit will issue a corresponding signal and the procedure will have been completed. If the target bacteria were present in or on the sample, then this will be immediately apparent. The procedure can be completed in a matter of minutes. The used element can be discarded. It is safe to dispose of the element after the test as any detected bacteria will have been killed by contact with the bacteriophage.

The element bearing the bacteriophage can include a reservoir of nutrient to facilitate multiplication of the bacteriophage on contact with the target bacteria. For example, the element might be a card with a groove or channel formed adjacent or extending from one edge, and in communication with a reservoir of nutrient. However, normally the nutrient and bacteriophage would be immobilised together on the element. Such an element could be formed with a plurality of grooves or channels charged with a different bacteriophage to target different bacteria, although it is not essential that grooves or channels are formed. The bacteriophage, with or without nutrient, can be in the form of deposits secured on the element, by a printing technique for example. The grooves, channels or deposits may also be colour coded.

The element may be produced in groups or sets, and a selection of elements or cards having differently charged grooves or channels can be provided for use in detecting different bacterial strains. The sensor can be designed to monitor the fluorescence from each channel or groove thereby determining not only where the bacteria is present, but identifying one or more of several different bacterial strains in what is essentially the same detection process. The elements can also bear information relating not only to the bacteriophage they carry, but also details of an individual or location which provides the sample under consideration, as well as other desired identification. A magnetic strip may be incorporated in the element for this purpose.

According to one aspect of the present invention, there is provided a bacterial detection sampling device comprising:
a sampling medium for receiving a bacterial sample; and
a plurality of bacteriophage located on or in the sampling medium, wherein each bacteriophage comprises a nucleic acid encoding a protein capable of emitting light at an output wavelength.

Conveniently, the nucleic acid encodes a fluorescing protein, the fluorescing protein being responsive to light of an input wavelength by emitting light of the output wavelength.

Preferably, the fluorescing protein comprises Green fluorescent protein (GFP); the input wavelength being 395 nm, and the output wavelength being 510 nm.

Alternatively, the nucleic acid encodes a chemiluminescing protein capable of emitting light at the output wavelength in the presence of a luminescent substrate.

Advantageously, the chemiluminescing protein is luciferase and the luminescent substrate is lucifern.

Conveniently, the bacteriophage are specific for infecting and/or lysing one strain of bacteria.

Preferably, the strain of bacteria is a strain of methicillin resistant Staphylococcus Aureus (MRSA).

Advantageously, the strain is MRSA strain 3, 15 or 16.

Alternatively, the strain of bacteria is a strain of Bacillus anthracis.

Advantageously, the sampling medium is a solid substrate.

Conveniently, the bacteriophage are immobilised on the substrate.

Preferably, the bacteriophage are immobilised on the substrate by a covalent bond.

Advantageously, the covalent bond between the bacteriophage and the substrate is supplemented by a coupling agent.

Preferably, the substrate comprises nylon or another polymer with amino or carboxy surface groups and the coupling agent is carbodiimide or glutaraldehyde; the substrate comprises cellulose or another hydroxyl-containing polymer and the coupling agent comprises vinylsulfonylethylene ether or triazine; or the substrate comprises polythene or similar polymer and the coupling agent comprises corona discharge or permanganate oxidation.

Conveniently, the bacteriophage is immobilised via its head group leaving the tail group free.

Preferably, the substrate comprises a plastics material.

Advantageously, the device further comprises an aqueous nutrient medium, preferably containing glucose.

Conveniently, the bacterial detection sampling device further comprises a receptacle for receiving the sampling medium.

Preferably, the receptacle is an ELISA plate.

Advantageously, the device comprises a plurality of bacteriophage strains, each strain being specific for infecting and/or lysing a different strain of bacteria and the bacteriophage of each strain comprise a nucleic acid encoding a protein capable of emitting light at a different output wavelength.

Conveniently, the device comprises two sheets connected via a hinge. Preferably, one of the sheets has a slide-covered aperture for receiving the bacterial sample. Advantageously, both sheets have slide-covered apertures, which are arranged so as to be aligned when the sheets are folded together at the hinge. Conveniently, at least one sheet is provided with an adhesive for sticking the two sheets together.

According to another aspect of the present invention, there is provided a bacteria detection device comprising:
a socket for receiving a bacterial detection sampling device according to any one of the preceding claims; and a light detector capable of detecting light at the output wavelength from the location of the socket.

Conveniently, the bacteria detection device further comprises a light source capable of emitting light at the input wavelength in the location of the socket.

Preferably, the bacteria detection device further comprises a user interface, in communication with the light detector, for providing an indication of the detection of light at the output wavelength.

Advantageously, the bacteria detection device further comprises a processor interposed between the light detector and the user interface, the processor being for calculating the change in intensity of light at the output wavelength detected over time and indicating the change in intensity via the user interface.

Conveniently, the light detector is capable of detecting light at a plurality of different output wavelengths.

Preferably, the bacteria detection device further comprises a bacterial detection sampling device as described above.

According to a further aspect of the present invention, there is provided the use of a bacteriophage for the detection of bacteria, wherein the bacteriophage is capable of binding to the bacteria and whereby a signal is produced in response to binding of the bacteriophage to the bacteria.

According to another aspect of the present invention, there is provided the use of a bacterial detection sampling device or a bacteria detection device of the invention for detecting bacteria in a sample.

According to yet another aspect of the present invention, there is provided a method of detecting bacteria in a sample comprising the steps of:
a) exposing the sample to bacteriophage each bacteriophage, comprising a nucleic acid encoding a protein capable of emitting light of an output wavelength, such that the bacteria in the sample are infected with the bacteriophage and the nucleic acid is expressed in the bacteria; and
b) detecting light emitted from the sample at the output wavelength, the detection of light indicating the presence of bacteria.

Conveniently, the nucleic acid encodes a fluorescing protein, the fluorescing protein being responsive to light at an input wavelength by emitting light at the output wavelength and wherein the method further comprises the step of exposing the sample to light at the input wavelength.

Alternatively, the nucleic acid encodes a chemiluminescent protein and the method further comprises the step of providing a chemiluminescent substrate in the sample.

Preferably, the bacteriophage is specific for a strain of bacteria and wherein the detection of light at the output wavelength indicates the presence of the strain.

Advantageously, the bacterial strain is a MRSA strain, preferably MRSA strain 3, 15 or 16.

Conveniently, the bacteriophage are the strain deposited as NCIMB 9563 and further comprise the nucleic acid encoding a protein capable of emitting light.

Conveniently, the bacterial strain is Bacillus anthracis.

Preferably, the bacteriophage are Bacillus anthracis phage Gamma and further comprise the nucleic acid encoding a protein capable of emitting light.

Preferably, the bacteriophage are part of a bacterial detection sampling device of the invention

Advantageously, step a) comprises the step of wiping the substrate relative to the sample.

Conveniently, step a) further comprises the step of growing the bacteria, after infection with the bacteriophage, in an aqueous nutrient medium, preferably glucose.

Preferably, step b) comprises detecting light at the output wavelength as the bacteriophage infects the bacteria, the detection of increasing intensity of light at the output wavelength indicating the presence of bacteria.

Advantageously, the sample is exposed to a plurality of strains of bacteriophage, each strain being specific for a different bacterial strain and the bacteriophage of each strain encoding a protein being capable of emitting light at a different output wavelength, the method further comprising the step of detecting the light emitted from the sample at each output wavelength, the detection of light at a wavelength indicating the presence of the corresponding strain of bacteria.

Conveniently, the method further comprises the step of killing the bacteria in the sample with the bacteriophage.

According to another aspect of the present invention, there is provided a bacteriophage having the genome of the strain deposited as NCIMB 9563 and further comprising a nucleic acid encoding a protein of emitting light.

The invention will now be described by way of example and with reference to the accompanying schematic drawings, wherein:

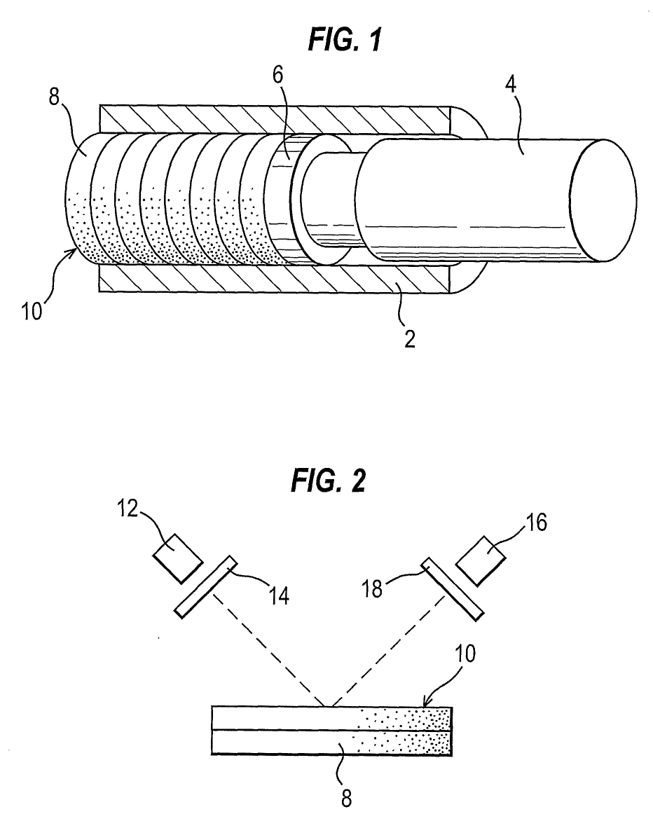

FIG. 1 is a part-sectional side elevation of a sampling device embodying the invention;

FIG. 2 illustrates how a plurality of bacteriophage can be examined to determine whether a targeted bacteria is amongst them;

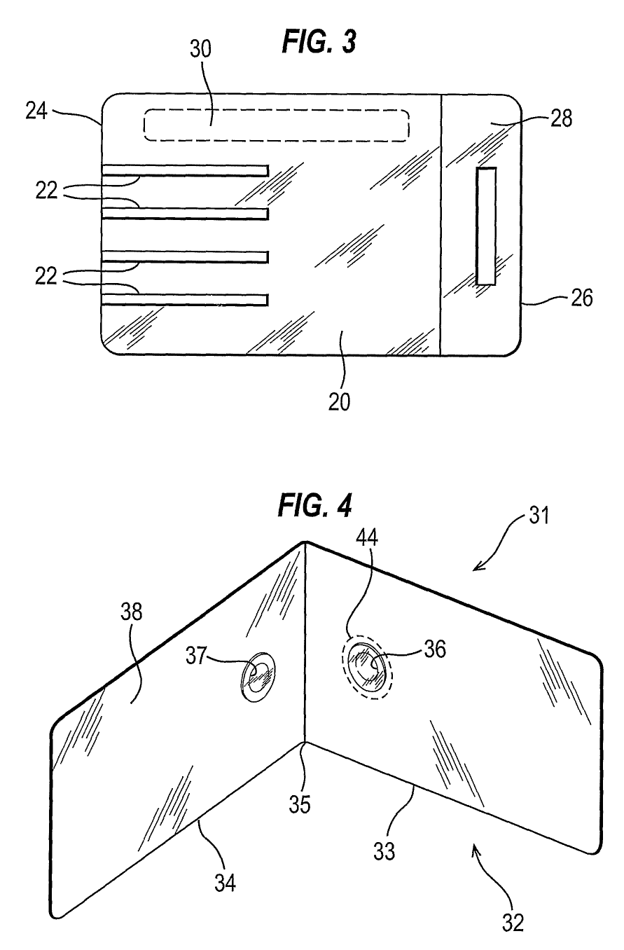

FIG. 3 is a plan view of a bacterial detection sampling device according to another embodiment of the present invention;

FIG. 4 is a perspective view of a bacterial detecting sampling device according to a further embodiment of the present invention; and

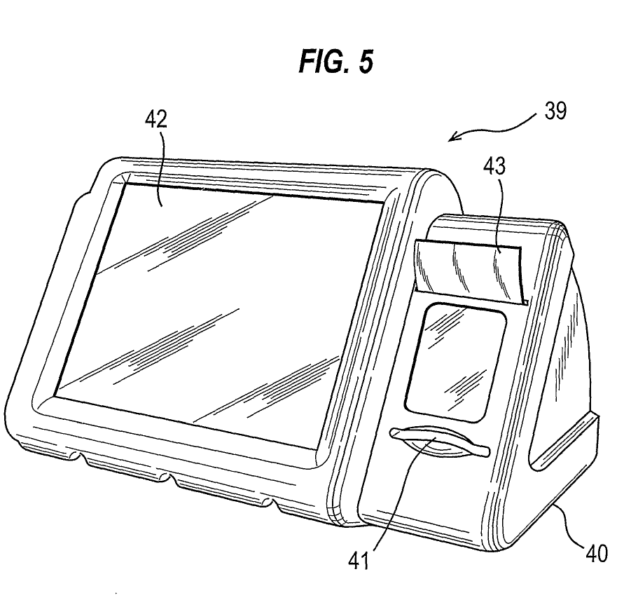

FIG. 5 is a perspective view of a bacteria detection device operable in conjunction with the embodiment shown in FIG. 4.

The device of FIG. 1 consists essentially of a tube 2 with a plunger 4 which can be pressed against a piston 6 to progressively push on a stack of pads or swabs 8 for contacting the sample to be examined. Each swab 8 may already carry the selected bacteriophage immobilised on the exposed or to be exposed surface 10 thereof. Alternatively, the bacteriophage can be applied to the surface 10 just prior to use. The device can then be used to wipe or otherwise contact the surface or environment of the sample under examination, so that the surface 10 is exposed to bacteria that might be present on or in the sample. The bacteriophage comprises a nucleic acid encoding a fluorescent protein (a suitable protein is Green fluorescent protein (GFP)) operably linked to a promoter. The bacteriophage would be selected for detecting a particular bacteria such as MRSA, or a strain or strains thereof. More specifically, the bacteriophage infects and may lyse a specific strain of bacteria. For example, the bacteriophage deposited with the National Collection of Industrial and Marine Bacteria under accession number 9563 (NCIMB 9563) is lytic for strains 2 and 12 to 17 of MRSA and is suitable to be used as the basis for such a bacteriophage. NCIMB 9563 must, of course, be adapted to include a promoter-linked GFP gene. As another example, the Bacillus anthracis phage Gamma (SEQ. ID NO: 1) is the typing phage for Bacillus anthracis and is also suitable as the basis for such a bacteriophage.

Each swab 8 is shown in the form of a disc, and it is typically formed of a plastics material and sterilised before use. The selected bacteriophage is preferably attached to the swab by covalent immobilisation, for example as discussed in International Patent Specification No: WO 03/093462 referred to above. The advantage of immobilising the phage is that their structure is thus stabilised which increases their longevity. Each swab could also be packaged with an aqueous nutrient medium that could support the growth of the target bacteria, or be moistened with such a solution prior to use. A suitable medium is methyl cellulose gel with 0.1% glucose but in other embodiments another cellulose derivative, galactomannon or other carbohydrate gel is used. It is important, however, that the gel is not, itself, fluorescent. It is also to be noted that the medium may be tailored for the bacteria to be detected. For example, for the detection of Bacillus anthracis using phage Gamma, a peptide mixture is provided in the medium.

When the swab 8 is wiped across or otherwise makes contact with the sample under examination, if the target bacteria is present then the bacteriophage fulfil their biological role and infect the bacteria, and themselves multiply before effectively destroying the bacteria by causing each bacterium to lyse or burst. During the phase of bacteriophage multiplication in each bacterium, the bacteriophage genome, including the nucleic acid encoding the fluorescent protein, is replicated and expressed. Thus each infected bacterium synthesises the fluorescent protein within it. Consequently, upon cell lysis, the fluorescent protein is released from the bacteria some of which may be incorporated into bacteriophage particles. The swab is then subject to optical examination, which stage is illustrated diagrammatically in FIG. 2.

As shown in FIG. 2, the surface 10 of the swab 8 is exposed to light from an LED or other source providing ultraviolet light. This is transmitted to the swab 8 through a suitable filter so that the light impinging on the surface 10 has the appropriate wavelength; for GFP the wavelength will be 395 nm. This exposure provokes any multiplied bacteriophage and, more specifically, the expressed fluorescent protein, on the surface 10 to emit light at the second selected wavelength; for GFP, 510 nm, and this fluorescence is detected by a photodiode, photomultiplier tube, charge coupled device or other detector 16, through a corresponding 510 nm filter 18. The amount of fluorescence received by the detector 16 is compared to that which would be emitted by the bacteriophage had they not multiplied; any significant increase of course indicating the presence of the target bacteria. If desired, the detector could be coupled to an appropriate processor to indicate either the presence or absence of bacteria, or to give an indication of contamination levels.

The advantage of the detection of the bacteria in this way is that is necessary for the bacteriophage to multiply in order for detection to occur and this, in turn, requires that the bacteria are live. Thus the present invention avoids any false positive results that could otherwise occur if dead bacteria were in the sample.

While in the above description of FIG. 2 reference is made to the swab 8 and its surface 10, it will be appreciated that the optical examination can be applied to the wiped or contacted sample, as an alternative or in addition to the swab, to take account of bacteria and bacteriophage transferring in both directions between the swab and the sample. Depending upon the nature of the swab or sample, the detector can be disposed on the opposite side thereof relative to the light source. It will also be appreciated that the components of the optical detector system can readily be incorporated in a handheld unit, which could be mounted in the same housing in which the device of FIG. 1 is held. Care, though, does have to be taken with regard to the use of the interference filters, which are required to separate the UV source from the Green detected light. Careful design of the optics and system geometry will also help to separate light from the source and from the fluorescence.

An element suitable for use in the above-described methods is illustrated in FIG. 3. It shows a card 20, typically the size of a credit card, and formed in a plastics material. On one surface are deposited four lines 22 of bacteriophage immobilised with a nutrient extending from a forward edge 24 of the card. Each of the four lines 22 carries bacteriophage with a specificity for a different strain of bacteria. Toward the rearward edge 26 there is ample space 28 for the card to be held by a user while the card is in use, and this space can bear some visible identification. Also shown in outline is a magnetic strip 30 for carrying additional information relating to the use of the card or the bacteriophage it carries.

In use, a sample suspected of carrying bacteria is wiped with a swab, and the swab then wiped over the lines 22 on the surface of the card 20. Nutrient in the grooves will enhance the growth or multiplication of any bacteriophage that has contacted a target bacteria with a subsequent increase in its fluorescent upon exposure to light of the requisite wavelength. The card bearing the potentially infected grooves is then introduced into an appropriately formed slot in a sensor unit (not shown) where the optical analysis is conducted. As each line 22 is associated with a particular strain of bacteria, the sensor can establish separately whether each of the selected bacterial strains is detected.

In one embodiment, instead of the provision of a stack of swabs as in the previous embodiment, a 96 well ELISA plate is provided, in each well there being located a nutrient sample (for example in a gel) containing the bacteriophage as in the previous embodiment. In use, a sample is obtained, for example, from wiping a swab along a surface and is then deposited in a well on the ELISA plate. Swabs can be wiped over different locations in a room or building, with each sample taken then being deposited in a different well on the ELISA plate. Once the bacteriophage has had sufficient time to infect any bacteria in the samples and to multiply the ELISA plate is examined using an ELISA plate reader. The advantage of this embodiment is that ELISA plate readers are widely available in hospitals and the like and thus the invention interfaces with existing hardware.

One particular example of a bacterial detection sampling device and a corresponding bacteria detection device is shown in FIGS. 4 and 5. Referring to FIG. 4, a bacterial detection sampling device 31 comprises a paper or card book 32 comprising first and second leaves 33, 34 connected at a hinge 35. The first and second leaves 33, 34 are each of the same size as a “credit card”.

At the end of the first leaf 33, adjacent to the hinge 35 is provided an aperture 36 in the first leaf, which is covered by a transparent slide. On the interior surface of the slide is provided a plurality of bacteriophage, each carrying a nucleic acid encoding GFP, under the control of a suitable promoter. The bacteriophage are covalently immobilised on the slide surfa. Covering the interior surface of the slide is provided a removable sticker 44 which protects the bacteriophage prior to use.

On the second leaf 34 is provided a second aperture 37 in a position which corresponds to the position of the first aperture 36 on the first leaf 33 in that, when the first and second leaves 33, 34 are pressed together, the two apertures 36, 37 are aligned. The second aperture 37 is also covered by a transparent slide. The remainder of the interior surface of the second leaf 34 is provided with an adhesive coating 38 which is covered by a wrapper (not shown).

Referring now to FIG. 5, a bacteria detection device 39 comprises a casing 40 in which is located a slot 41 of a size suitable for receiving the card 32. The bacteria detection device 39 also comprises a control screen 42 for providing input and receiving output from the device as well as a paper printer outlet 43. Within the casing 40 there is provided a source of ultraviolet light and a fluorescent detector (not shown) which operate upon the same principle as the device shown in FIG. 2. The source of ultraviolet light may be controlled using the control panel 42 and the results of the fluorescence detector may be observed in the control screen 42.

In use, a sample is collected on a swab, for example by wiping the swab on a surface in a hospital. The sticker 44 is removed from the first leaf 33 and the sample is deposited on the interior side of the slide in the first aperture 36. The wrapper covering the adhesive surface 38 is then also removed and disposed of and the first and second leaves 33, 34 are pressed together and secured in position by virtue of the adhesive surface 38. This prevents the escape of any hazardous material in the sample and also prevents the ingress of any matter which could contaminate the sample. Because of the positioning of the first and second apertures 36, 37, the sample is visible from either side of the card 32.

Subsequently the sample is left for a period of time to allow the bacteriophage to infect any bacteria in the sample and multiply within them.

The card 32 is then inserted into the slot 41 of the bacteria detection device 39, which is activated using the control panel 42. Ultraviolet light is then directed on the card 32 and, more specifically, through the first and second apertures 36, 37. At the same time, any fluorescent light emitted from the sample is detected by the fluorescence detector and, if any is detected, then the intensity thereof is reported on the control panel 42. Thus the control panel 42 provides an indication as to the presence or absence of bacteria in the sample, which the bacteriophage is capable of infecting.

In a variation of the embodiment shown in FIG. 1, instead of providing identical bacteriophage on the swab 8, a plurality of different bacteriophage strains are provided. Each bacteriophage strain is specific for a different strain of bacteria. For example, one bacteriophage strain is NCIMB 9563 which is lytic for certain strains of MRSA and a second strain of bacteriophage is Bacillus anthracis phage Gamma which is specific for Bacillus anthracis strains. Furthermore, each strain of bacteriophage comprises a nucleic acid encoding a protein which fluoresces at a different wavelength. The swab 8 is used as described above but, during detection, both emission wavelengths are observed and the presence or absence of either strain of bacteria can then be determined simultaneously by detecting the presence or absence of light emitted at either or both wavelengths.

While the above-described embodiments have been exemplified with green fluorescent protein as the fluorescing protein, it is to be understood that many other different types of fluorescing proteins are available. Examples of these are reef coral fluorescent proteins, which are available under the trade name Living Colors.

It is also to be understood that in further embodiments of the present invention, the bacteriophage comprises a nucleic acid encoding a different type of light emitting protein from a fluorescing protein. In particular, the protein may be chemiluminescent or phosphorescent. A particular example of a suitable chemiluminescent protein is luciferase. This 61 KDa enzyme catalyzes a two-step oxidation reaction to yield light, usually in the green to yellow spectrum, in the presence of a luminescent substrate (e.g. luciferin) and ATP. In these embodiments, the swab 8 also comprises a supply of luminescent substrate and ATP so that, if luciferase is released by lysed cells, it is able to emit light.

The molecular biology which is required to produce the components of the invention will now be described. In order to produce a suitable bacteriophage containing a nucleic acid which encodes a light emitting protein, a suitable strain-specific bacteriophage is first selected (e.g. NCIMB 9563 or Bacillus anthracis). DNA is extracted from the bacteriophages and purified by caesium chloride gradient centrifugation. Phage DNA is digested into suitable size fragments and then cloned into an E-coli plasmid. A plasmid containing suitable phage sequences flanking the nucleic acid encoding the light emitting protein is also constructed. This plasmid is incorporated into a shuttle vector and by a double crossover, the gene encoding the light emitting protein is introduced into the corresponding position in the phage genome.

An alternative approach to obtaining suitable bacteriophage is to use a transposon containing the light emitting protein gene, which is then inserted randomly into a number of bacteriophages. The phage are then multiplied in E-coli and colonies expressing the light emitting protein are selected. The recombinant phage DNA is isolated and incorporated into a suitable host bacterium (e.g. if the starting bacteriophage is NCIMB 9563 then the host strain is Staphylococcus Aureus) Plaques containing suitable bacteriophage are then selected.

The following references provide some useful discussion of the use of Green Fluorescent Protein in bacteriophage for the detection of bacteria:
1. Use of bioluminescent Salmonella for assessing the efficiency of constructed phage-based biosorbent. W. Sun, L Brovko, and M Griffiths J. Ind. Micro & Biotech. 2000 25 273-275.
2. Rapid Detection of Escherichia coli 157:H7 by using Green Fluorescent Protein-labeled PP01 Bateriophage. Masahito Oda, Masatomo Morita Hajime Unno and Yasunori Tanji Appl. Environ, Micro 2004 (Jan) 527-534.
3 Nachweis Und Identifikation von Bakterienstammen (Detection and Identification of bacterial strains) 01/09370 A2 Miller, Stefan.
4 The Molecular Structure of Green Fluorescent Protein. Yang F et al Nature Biotechnology 14, 1246-1251 (1996).

The attached text file, entitled “SEQUENCE LISTING,” created on Aug. 30, 2007, size 47,647 bytes, is incorporated by reference herein.

1. A bacterial detection sampling device comprising:
a sampling medium for receiving a bacterial sample; and
a plurality of bacteriophage located on or in the sampling medium, wherein each bacteriophage comprises a nucleic acid encoding a protein capable of emitting light at an output wavelength.

2. A bacterial detection sampling device according to claim 1 wherein the nucleic acid encodes a fluorescing protein, the fluorescing protein being responsive to light of an input wavelength by emitting light of the output wavelength.

3. A bacterial detection sampling device according to claim 2 wherein the fluorescing protein comprises Green fluorescent protein (GFP); the input wavelength being 395 nm, and the output wavelength being 510 nm.

4. A bacterial detection sampling device according to claim 1 wherein the nucleic acid encodes a chemiluminescing protein capable of emitting light at the output wavelength in the presence of a luminescent substrate.

5. A bacterial detection sampling device according to claim 4, wherein the chemiluminescing protein is luciferase and the luminescent substrate is lucifern.

6. A bacterial detection sampling device according to claim 1 wherein the bacteriophage are specific for infecting and/or lysing one strain of bacteria.

7. A bacterial detection sampling device according to claim 6 wherein the strain of bacteria is a strain of methicillin resistant Staphylococcus Aureus (MRSA).

8. A bacterial detection sampling device according to claim 7 wherein the strain is selected from the group consisting of MRSA strains 3, 15 and 16.

9. A bacterial detection sampling device according to claim 1, wherein the bacteriophage are the strain deposited as NCIMB 9563 and further comprise the nucleic acid encoding a protein capable of emitting light.

10. A bacterial detection sampling device according to claim 1, wherein the strain of bacteria is a strain of Bacillus anthracis.

11. A bacterial detection sampling device according to claim 10 wherein the bacteriophage are Bacillus anthracis phage Gamma and further comprise the nucleic acid encoding a protein capable of emitting light.

12. A bacterial detection sampling device according to claim 1 wherein the sampling medium is a solid substrate.

13. A bacterial detection sampling device according to claim 12 wherein the bacteriophage are immobilised on the substrate.

14. A bacterial detection sampling device according to claim 13 wherein the bacteriophage are immobilised on the substrate by a covalent bond.

15. A bacterial detection sampling device according to claim 14 wherein the covalent bond between the bacteriophage and the substrate is supplemented by a coupling agent.

16. (canceled)

17. A bacterial detection sampling device according to claim 13 wherein the bacteriophage is immobilised via its head group leaving the tail group free.

18. A bacterial detection sampling device according to claim 12 wherein the substrate comprises a plastics material.

19. A bacterial detection sampling device according to claim 1 further comprising an aqueous nutrient medium.

20. A bacterial detection sampling device according to claim 1 further comprising a receptacle for receiving the sampling medium.

21. A bacterial detection sampling device according to claim 20 wherein the receptacle is an ELISA plate.

22. A bacterial detection sampling device according to claim 1 comprising a plurality of bacteriophage strains, each strain being specific for infecting and/or lysing a different strain of bacteria and the bacteriophage of each strain comprise a nucleic acid encoding a protein capable of emitting light at a different output wavelength.

23. A bacteria detection device comprising:
a socket for receiving a bacterial detection sampling device according to claim 1 and a light detector capable of detecting light at the output wavelength from the location of the socket.

24. A bacteria detection device according to claim 23, wherein the nucleic acid of the bacteriophage of the bacterial detection sampling device encodes a fluorescing protein, the fluorescing protein being responsive to light of an input wavelength by emitting light of the output wavelength and wherein the bacterial detection device further comprises a light source capable of emitting light at the input wavelength in the location of the socket.

25. A bacteria detection device according to claim 23 further comprising a user interface, in communication with the light detector, for providing an indication of the detection of light at the output wavelength.

26. A bacteria detection device according to claim 25 further comprising a processor interposed between the light detector and the user interface, the processor being for calculating the change in intensity of light at the output wavelength detected over time and indicating the change in intensity via the user interface.

27. A bacteria detection device according to claim 23 wherein the light detector is capable of detecting light at a plurality of different output wavelengths.

28. A bacteria detection device according to claim 23 further comprising a bacterial detection sampling device according to claim 1.

29. A method of using bacteriophage for the detection of bacteria, wherein the bacteriophage is capable of binding to the bacteria and whereby a signal is produced in response to binding of the bacteriophage to the bacteria.

30. A method of using a bacterial detection sampling device according to claim 1 for detecting bacteria in a sample.

31. A method of detecting bacteria in a sample comprising the steps of:
a) exposing the sample to bacteriophage each bacteriophage comprising a nucleic acid encoding a protein capable of emitting light of an output wavelength, such that the bacteria in the sample are infected with the bacteriophage and the nucleic acid is expressed in the bacteria; and
b) detecting light emitted from the sample at the output wavelength, the detection of light indicating the presence of bacteria.

32. A method according to claim 31 wherein the nucleic acid encodes a fluorescing protein, the fluorescing protein being responsive to light at an input wavelength by emitting light at the output wavelength and wherein the method further comprises the step of exposing the sample to light at the input wavelength.

33. A method according to claim 31 wherein the nucleic acid encodes a chemiluminescent protein and the method further comprises the step of providing a chemiluminescent substrate in the sample.

34. A method according to claim 31 wherein the bacteriophage is specific for a strain of bacteria and wherein the detection of light at the output wavelength indicates the presence of the strain.

35. A method according to claim 34 wherein the strain is a MRSA strain.

36. A method according to claim 34 wherein the strain is Bacillus anthracis.

37. A method according to claim 31 wherein the bacteriophage are part of a bacterial detection sampling device according to claim 1.

38. A method according to claim 37 wherein step a) comprises the step of wiping the substrate relative to the sample.

39. A method according to claim 31 wherein step a) further comprises the step of growing the bacteria, after infection with the bacteriophage, in an aqueous nutrient medium.

40. A method according to claim 31 wherein step b) comprises detecting light at the output wavelength as the bacteriophage infects the bacteria, the detection of increasing intensity of light at the output wavelength indicating the presence of bacteria.

41. A method according to claim 31 wherein the sample is exposed to a plurality of strains of bacteriophage, each strain being specific for a different bacterial strain and the bacteriophage of each strain encoding a protein being capable of emitting light at a different output wavelength, the method further comprising the step of detecting the light emitted from the sample at each output wavelength, the detection of light at a wavelength indicating the presence of the corresponding strain of bacteria.

42. A method according to claim 31 further comprising the step of killing the bacteria in the sample with the bacteriophage.

43. A bacteriophage having the genome of the strain deposited as NCIMB 9563 and further comprising a nucleic acid encoding a protein capable of emitting light.

44. A bacterial detection sampling device according to claim 15 wherein the substrate is selected from the group consisting of nylon polymer with amino surface groups and a polymer with carboxy surface groups and the coupling agent is selected from carbodiimide and glutaraldehyde.

45. A bacterial detection sampling device according to claim 15 wherein the substrate is selected from the group consisting of cellulose and a hydroxyl-containing polymer and the coupling agent is selected from the group consisting of vinylsulfonylethylene ether and triazine.

46. A bacterial detection sampling device according to claim 15 wherein the substrate is selected from the group consisting of polythene and polymer similar to polythene and the coupling agent is selected from the group consisting of corona discharge and permanganate oxidation.

47. A bacterial detection sampling device according to claim 19 wherein the aqueous nutrient medium contains glucose.

48. A method of using a bacteria detection device according to claim 23 for detecting bacteria in a sample.

49. A method according to claim 35 wherein the MRSA strain is selected from the group consisting of MRSA strains 3, 15 and 16.

50. A method according to claim 39 wherein the aqueous nutrient medium comprises glucose.


SEQUENCE LISTING (1 sequence)

SEQ ID NO: 1 - DNA, 37253 bp, Bacillus anthracis Gamma Phage
cgccgccccctcaacttcgcagaaaaatccgtttttgcatatttttttaagggggtgtaatcatggctggaagaaataaacaaccactctctgttatacagggaaaaggtagatcaaatcacattacaaaaagtgagaaaaacagacgagaaaaacaagaagaagcattgcgggggcatactgataaaattgaagctccttcttatttgactgcagcacaaaaaaaggaattcgatactttagctgctgaattagtcagattgaaaattttcagtaacttagatgttgacagtttagcaaggtacgttgattctaaagaccaatatataaaaatggttcgtctgctaagaaaaacaaaaccttcagatgactttaaattgtattctcaaatgcaaagaagtaaaaatcttttattcaatgaatgccgttcttcagctagtgatttaggtttgaccattacatcccgcttaaaattagttattccagaagtagatacttcacaacaaaagcaaagtgaagcgcaaaagcgttttggtgatcgtatatgaactggataatggaacgggtttttgcatattgcgaggacattttaaacggcaagataaatagttgtaaaaaacatcgttgggccattgaacgatttataagggattatgaggagtgtcaaagtgaagacagtcctttttattttgatggagagatagcggaggatttttactggtttgcaaaggaatttaagcacgttgaagggattttggcaggtgaatccgtagaattaactgattttcaattgtttctagcggctaatattttcggattcaaaaagaaaataaatggagcaaggcgatttagaaaggtttttattcagttagcgcgtaaaaatgctaaatctcagtttcttgctattgtagcagctttttgtacatttcttggagacgaaaaacaacgggcttatattgctggatggacaagagaccaatcatctgaagtttatgaagctgtaaaaacagggattagttctagtgaattgttagaaggtaaatggaaagaggcttatagtaccattgaaatatttaagaatggttcagttgtcgttccactttcaaaagaagctagaaaaactggtgatggtaaaaacccgtctcttggaattgtcgatgaatatcatgcacatgaaactgatgaaatttatgacgttttatcgtctggtatggtggcaaggaaagagccgttaatgtttatcataacaacagctggtttcgacttatcaagaccttgttatagagagtatgagtatgtcagtgacatcttagacccgtcaaaaaatgtagaaaacgatgattatttcgttatgatctgtgaattggaaaagaacgatgatatcaaagatgagtcgaattggataaaagcaaacccaatcgtagctacatatgaagaaggtttggaaggtatacgttcagatttgaaggttgctcttgatagacctgaaaagatgagggcttttttaaccaaaaacatgaatatttgggtcgataaaaaggacaacggatacatggatatgtcaaaatggcaaaaatgcgaagtagatacctttgatttttcaggtgcgactctttggataggtggcgacttatcaatgacaacagatttaactagtgtcggttgggttggaatggacgatgaaggtgattttattgttggacaacattcatttatgcctgaagcacgtttgaaagaaaagatggccatagataaggtgcgttatgatttatgggccgaacaagggtatttaactttaacgcctggtgaaatggttgattatacaattgttgagtcttggatagaaaacttttcaaaagacaaagaaattcaagagtttgattacgataaatggaatgcgttacatctagcacaaaatttagagaataaagggttcgtttgtgtagaaatccctcaaaggattgctaatttatccattccgactaaaaattttcgagaaaaagtatacgaaaagaaagttaaacataatggagatccagtccttttttgggcgcttaataatgctgttgttaaaatggatgatcaggaaaacattatgatttcgaaaaaaataagtaaaaatcgtattgatccagcagcagcggtcttaaatgcattttctagggctatgtatggagcaagtgtcaggtttgatgtatctgaatttgcaaataaagactttctaggcaagttatggaactagggagggggtgaacatgtgaagatagtggattctgttaaaaagttctttaattttgaaaaacgccaaacgtcgcaggtaatagagttgaataaagacgatgaaaaattattagaatggctagggatttctccaagtactattagcgttaaaggaaaaaatgctttaaaagttgctacagtctttgcttgtatcaaaatactatctgaatccgtatcaaagttaccgttgaaaatttatcaggaagatgaatatggaatccaacgcggtacaaagcattatctcaacaatttactgagactaaggcctaacccgtatatgtccagtatgaactttttcggatcattagaagctcaaaaaaatttatatggcaatagctacgctaacatagagtttgatagaaaaggtaaagtccaagcgttatggccgatagatgcttctaaagtgacagtatacattgatgacgttggtttattaaattccaaaactaaaatgtggtatgtagtaaatacgggtggacaacaaagagtgttaaagccagaagagatactgcactttaaaaacggaataactcttgatggtcttgtcggtgttcctacaatggaatatttaaagtctacattagaaaattcagcttcagctgataaattcataaataatttttacaaacaagggttacaggtaaagggattagttcaatatgtcggtgatttaaatgaagatgcgaaaaaggttttccgagaaaatttcgaatcaatgtctagcggtcttcaaaatagccatcgtattgcattaatgccagtaggatatcaatttcaacctatttcattaaatatgtcagatgctcaatttctcgaaaataccgaacttactattaggcaaatcgctactgcattcggcattaaaatgcatcaattaaatgatttgagtaaagcgactttaaataatattgagcagcagcaacaacaattctataccgatacattacaagcgactttaacaatgtatgagcaagaaatgacgtataagctatttttagacagtgagttggataaggggttttattcaaaattcaatgtagacgctattttaagagcggatatcaaaacgagatatgaagcttacagaacgggtattcaaggcggtttccttaaacctaacgaagctagaagtaaagaagatttaccaccagaagctggtggggatcgtttacttgttaatggaaatatgttgccgattgatatggctggacaggcatatttgaagggaggtgatactaatggagaagtcagcaaagaaggaaatgaaggaaattagagctttgccaatgactattgaagtccgtgaagttaatgaggacgagggaaaacgaacaatttcgggatcgataaaatataacaatgaaagtgccgaaatgcgtgactggtggggcgatactttcgtagaagagattgctgagggagcttttgatgaaagtttaaaagttcgtgatgttgtaggtttatggtctcacgacacatctcaagtattaggaaatactaaaagtaaaactttacgaatcgaaaatgacaagaaagaattacgatttgaattagatattcctaatacaactgttgggaatgacgcatgggaattaattaagcgtggagatgttgatggagtttcttttgggatgaaggttacaaaagacaaatggtcatcggaagaacgtgaaaatggaaagctttataagcgttcgattttaaatgctgaactatatgaaatatcaccggttgcattccctgcatatccaacgaatgaagtaagtgtacgttcattggatgattttaaagctggagaaaagcgagtagctgatgagtttaggaaaagaaaactacaaatcgaactagagcttatataaggctctttttttattgataaatttaaggagtgatttgaatgtcaaaagaattacgtgaattattagctaagttagaagggaaaaaggaagaagtacgctctcttatgggagaagataaagtggcagaagcagaacaaatgatggaagaagtgcgatcacttcagaaaaaaattgatttacaacgctcattagatgaagcagaaacggaagaacgaaataatggaagagaagttgaaacacgtaatgtagatggtgaaatggaataccgcgatgtgtttatgaaagcattacgcaataaaccattaaatgctgaagaacgtgaatttcttgaggatgatttagaacaacgtgccatgtcaggattaactggggaagatggaggacttgtcatccctcaagatattcaaacgcaaatcaatgaattagctcgttcatttgatgcgcttgagcaatatgtaactgttgaaccagtgcgtacacgttcaggatcacgagtattagagaaaaattcagatatgattccgtttgctgaaatcactgaaatgggtgaaattccagaaactgataatccgaaattttcaaatgtacaatatgcagtgaaggacagagcaggtattttaccgttatctcgttcattacttcaagatagtgatcaaaacatcctaaagtatgtgactaaatggctaggtaagaaatctaaagttacacgtaatgtgttaatcttgggcgtaattgaaaagttaacaaaacaagcaatcaaatctctggatgatattaaagatgtattaaatgttaaattagacccagcgatttctccgaatgcgattttacttacaaaccaagatggatttaattatttagacaaattaaaagataaagacggaaaatatattttacagtcagatccaacgcaaaaaaacaaaaaactatttgctggtactaatccagtcgttgttgtttcgaatcgtttcttaaaatcaaagggaactacagctaaaaaagcgccacttattattggtgatttaaaagaagctattgttttatttaaacgtgaagatatggaactggcttctacagatgtaggtggtaaagcattcactcgtaatacattagatttacgcgcaattcaacgtgatgatgtgcaaatgtgggataatgaagcagcagtttacggagaaatcgatttaagcgctcctgttgaacaacctcaagggtaaactaaggaggcatttgaatgcttgttaccttagaagaagctaaagaatggattcgagtggacggagacgatgacccaactatcactatgttaattaaagcggctgaattatatatttacaaagcaactggcaaaacatttactcaaacaaatgaagatgctaagttgctttgtttatttctggtggctgattggtacggaaatcgactacttgtaggtgaaaaagccagtgaaaaaatcagaaccattgttcagagtatgatattacagctccaatatgcttcagagcctcaggaggaaagaaaatgaatcctgcaaaattagataaacggcttacatttcaagtaaaagatgaaaatgcaaaagggcctgacggtgatccgatagatggatataaagatgcttttaccgtatggggctcttttgtttatttaaagggaaggaaatactttgaggcagcagctgctaatagtgaggttcaaggagaaacagaaatcagaaatcgggatgatgtaagtgcagatatgaaaattaagtacaaaaacgtgatttatgatattgtttccgttattccaactcaagatcatactttattaatcatgtggaaacgtggtgaaatgaatggctgatggtatagatttagatttattaggatttgatcgtttagttactgaattagaccaaatggggttacggggagagaaaattgaagataaagctcttgcagctggtggtgaacctattcgtaaagccattgcagaacgagcgccaagaagcccaagccccaaaaaacgatctaaaagtgaaccgtggcgtacagggcaacatggtgcagaccagataaaagtaacaaaagctaaacttgaaggtggaataaaaacagtaaaaataggtcttaataaagcggatcgttccccgtggttctatttaaagttccatgaatggggtacatccaaaatgccagcacatccatttatagagccgggttttaatgcttcaaaagcggaagctgtacgtgctatgacagatattttaaagaacgaaatgaggttggatttgtgataaatttaagacctgatattttacaagctcttgagaatgatcaagagcttgtttcattgttgggtgggaaacgaatttattaccgtaaagcaaagaaggcagaagagtttccgcgaattacgtattttgaattagacaataggccagatggatttgcagataatcaagagattgaaagtgaaatcttgtttcaagttgatgtttgggcaaagagtagtacaacagcaatccatcaaaaagtgaatgaaatcatgaaaagaattggtttctcacgctatgcggttgctgatttatatgaagaggatacacaaatatttcattatgcgatgagattcgcaaaaggagtggaattataaatggctggagaagttgtaagaattagttcaacggttggtgtagacaaccttgtatatgcgaaagttttacaagatgattcgtctgctattaaatatacagatgtaaagaaaatggaaggtgctgtaaaggttaaattaactaaaaaagtagcttctgaggttatgtggagcgataacagaaaatcagagattgcagaatctgatggcgaaactgaagtggagattgaggttcgaggactttcactttctacaaaggctgacattgaagggtttccagaagtaaaagatggcgttttagatgagaaacgtgaaggtgagaaaccatatttagctattggtttccgattcttaaaagctaatgataagtatcgatatgtttggttattaaaagggaaactttcacaagaggaagaagaagctgaaacgaaaaaagacaaaccgaacttccaaacaacaaaattgaaaggttcctttattgaacgtgattttgatgatagaacgaaatttacagcagatgaagatgaaccaacgttcacaaaattagttggagataattggtttaataaagtatatgaaaaaccagtgacacaaccaccagcaggaaagtaagagggagcaaaagctctctcttttttattaaatttaggagggaaaaactatgaaattaacattaatgattaataaagaaaaacaaacttttaatatgccagaatttattccagcccgccttattcgtcaggctcctgaacttgctgaaattccaaacaatcctggtccagaagatatggataaaatggttcaattcgtagtgaaagtttatgatggtcaatttacattagatcagtattgggatggtgttgatgcccgtaaattcttatcgacaacttcagatgtaattaacgcaattataaatgaaacagtggaagcagcagggggtagtactgaatcaggagaagaagaaaacccaaacgcatagagggaggagggctaacgttcagtgagtttatggacgagctctacctctctttattgcgacaagggtacaaacaccatcacattgataatgagatggatatttggcattatttgagacttaatcgaaaaatgcatgaaaacggaaatgaaaattacgaaggctccaattcaaatgaaatagaagtgccagcggaaaacattatttaacgagggaggtgagactatggcgaatgaaataaataatctagtcgttagactttcccttgataacgtaaatttcagacaaggtatctcgaattcaggtcgtgcagtcaggacgttacagaatgaattgaaatctgtaagtacaggaatgggcggttttgctaacgctagtcagcaaacacaagcgaaaatgaatacactcagtaggctcattgatgcgcaaaaagagaaagttaaagcgttacgacaagcctatgatcaaaataaggctaaattaggtgaaaatgatgcagcaacccagcgatatgcttcgcaagttaataaggcagttgctgatttaaatagatttgaaaatgaattaaagcaagtaaaccgtcaagctgaacaaaaagggatggataagttaaacaactctttaaaatccctacaagctgaatttcagtctattacaacaggtatgggcggtttttctaatgcgacagaacaaacaagggctaaagtagatgttttatcccgtatggtagataaacaaaaagagaagattagggaacttcaacaagcctataatcgtgctaaaacagaagaaggcgaagcgagtcaatcagcacaaagatacgctgaacaaattcatcgggcaacagctgaactgaatcgatttgaaactggattacagcagtcaaatcgtgaattagaacagcaagggaatcgcctattgaacttcggaaatcgcatggagacattaggtaatcatttgcaaaatgccggaatgcagatcggcatggtatttggtggtatgacttacgcaataggtcggggcttaaaatcagcaatcactgaatcaatgaattttgagcaacagatggccaatgtaaaagctgtttctggatctactggagcagaaatgaaaaagttaagtgaattggctgttaatatgggagaaacaacaaaatactccagtgttcaagcaggtcaaggtatcgaggaattaataaaggctggtgttagcttacaagatattattaacggcggattggcaggtgcccttaacttagcgacggcaggggaattagagttaggtgaagcagccgaaattgcttccacagctctgaatgcatttaaagcagaccatctttcagttgcggatgcagccaatattttatctggtgcagccaatgcttccgcaactgatgtaagagagttaaaatatggactttcagcttcatcagcagtagcagcgggagccggaatgacgtttaaggatacagctacaactttagcggtatttgcacaaaatggtcttaagggatcagatgcaggtacatctttaaaaacaatgttaatgaggttaaatccttcaacaaaagaagcatataacaaaatgagagatttaggacttattacttataatgcacaggcaggttttgatttcttagttaaaaacggtattcaaccagcttccagaaatgtaggggatatagaagtagctttagaacaatatgtaatgaaaacagaaggtgtaacgaaatggaatgataaatgtgatacaacgtttcgcgaattagcaacaagttcggcatttttatcatcaaaattctatgatcaacaggggcatattcaaagtctagaaaatatttcaggtacacttcatgaatcgatgaaagatttaacagaccaacaacgaagtatggctctggaaacattatttggttccgatgctgtacgtggtgcgactatcttgtttaaagaaggcgccaaaggtgtcaatgaaatgtgggattccatgtcaaaggttacagcagctgatgtagcagcgaccaaaattgatactttaaagggacgacttacattactagattcagcgttttccacaatgaaaaagacaattggtgatgcactagctccagtagttagtgtttttgttgctggtttacaaaaacttgttgatggattcaactctttacctggaccagtacaaaaggcaatagcaattacaggtggtatcgtccttgctttaacagctgtggctacagcaataggtgtggttttagcagcgtttggaatgattgcttcaggaattggttctttatctcttgctttagcatcagtcggtgggattgctggaattgcggctggagcagttggattcttaggaagcgcgcttgcggttttaacagggccaattggtctagtagcagcggctcttatcggaactggtgttgttgcatataaagcatatcaaaaagcgactgaagacagtatcgcatcagtagaccgctttgctacaaatacagaagggaaagtaagctcctcaacaaagaaggttcttggcgagtatttcaagctgtccgatggtattagacaaaagttaactgaaattagattgaaccatgaagtaataacagaagaacagtcgcaaaagttgattggtcaatatgacaaattagctaatacaatcattgaaaaaaccaacgcaaggcagcaaaaagaaattgaagggcttaaaaagttctttgctgattcgtatgtattaaccgctgaagaagagaacaaacgaatcgaacagttaaatcagcactatgaacaagaaaagctaaaaacgcaagaaaaagaaaataaaattaaagagatcttacaaacagcggctagagaaaacagagaattaacgacatccgaacgtatctctttacaagcattgcaggatgaaatggacagagttgctgttgagcatatgtctaaaaatcaaatggagcagaaggttattcttgaaaatatgcgtgtgcaggctagtgaaatttcagctagacaggcagcggaagttgtagagaatagcgccaaagcaagagataaagttattgaagatgcgaaaaagacccgtgatgaaaaaattgcagaggcgattcgccaacgtgatgaaaataaaacaatcactgctgatgaagcgaacgcaatcattgcagaggcaaaacgtcaatatgatagtacagtttctacagctcgagataaacataaagaaattgtgagtgaagcaaaagcgcaagctggtgaacatgcaaatcaggtagattgggaaactggccaagtaaaatcgaaatatcaagctatgaaagacgatgttattcgaaaaatgaaagaaatgtggtcggacgttaccaacaaatatgaagatatgaaaaactctgcaagcaacaaagtagaggagataaaaaatacagtttcaagaaaatttgaagagcagaaaaaagctgttactgataagatgtcagaaataaaaagtagtattgaagataagtggaatacagttgaaaagtttttcagttctataaatttacgttccatcggtaaatcaatcatagaagggcttggcaagggaatagatgacgcttcaggaggtctgtttagtaaggctgcggaaattgcaagtgatattaagaagactatttctggagcattagaaattaacagtccgtctaaagtgatgattccagtcggtagcgcagttccagaaggtgttggggttggtatggataagggaaaacgatttgttgtggatgcagcaaaaaatgtagtcggaactgttaagaaacaaatggggaatatgccatctgtttttgattttggattccaaacaaatcaatatagtatcccgcaaaatacatttagcgatttcagtggatatatgcaaccgcaattatcttataacaatccatctatggcaaaaacaatattcccaaatagaccaggtggagaacaagaactgaatttaaccgtaaacatgactaatgttttagatggaaaagagcttgcaaacggaagttacacctatactacaaaacttcaaaatcgtgaacaaaaaagaagagcggaattttaagggtggtgagcacgttggggaaacttagttttacttttaataatattagaaaagattatattcaaatgctagttggaagaaaacgtccttcatgggctccagtaaaaagaagattagtaagagtccctcatcgcgcaggggctcttttacttaatacagaaacggaggaacgtcgtattgacgttcctcttgttattaaagcgaaaaaagatatggcagatttacaaaagttaaaagaagatttagcggattggttatatacagagcaacctgctgaacttatttttgatgatgagttagacaggacttatttatcattaattgatggttctgtcgatttggacgaaatagtcaatagaggtaaaggtgttattacttttgtttgtccaatgccgtataaattagggaaaatcaatactcacaaatttacgcaagagtggtctacagaaacaacttcttattttactaataaaggaagtgtagaagctccagcattaattgaaatgacagtgaaaaaaccaagtacctttttagatgtatggtttggagagtatccgcataatcgtgattatttcagaataggctaccctctgactgtggaagaaaccacggtacaagaacgagaaagagtcatgtgggatgaaatggctactcctataggatggacacctgttactggacaattcgaggagatgaaagggacaggtagttttaaatcaagaggtggtcatgcactatattgtgaagattacggaaaagagacaggattctacggtgctatagccaagaaaaacattccgggcggcccattacaagacttcgaaatggaggcatgggtgactttaaagtccaaaaacataagcgaaatgggacgtgttgaagttcttcttttagatgagacgagtaacgtgatatcccgcatcaatatgaatgatctatatgcgaccgctgaaattacaagggcgcatatgacaattggaaatagcggaacacccaatagttttcgaaaattagttgatacaagtggattttattcgacaacatttaaccaattccgagggcgtttacgtattgctaggcgggggaaggtgtggtctgtatatgtggctaaatttatagatggtacagaaaaagatggagcttcacttgtagaacgttggattgatgaaacaggaaatccgatgacagaacgtaaaattgcacaagttatgattgcgatttgcaagtgggataatcatcaacctattaacgaaatgcaaattgatgatttaaaaatttggaaggtaaacaaagttccatctaatgcacaaccatatatctttgatactggagataaaattgttatcgatactgagaaaagtcttgtcacgatcaatggggagaaagcaatcaatataaaagaaatctttagtaattttcctatcgtaatacgtggtgaaaatcgtatcgatataatgccacctgatgtaaatgcaacaatcagttatagggagagatatagatgagaacaccaagcgggattttgcatgttgtggattttaaaacggatcaaatcgtcgcagctatccagccagaggactattgggatgacaaacggcattgggaactaaaaaataacattgacatgttggatttcaccgcttttgatggaacagaccatgcagttacgttacaacaacagaatcttgttttaaaagaagttcgcgatggaagaatcgtaccgtatgttattacagagactgaaaaaaattccgataaacgatccattaccacatacgcttcaggagcttggattcaaattgctaaatcaggggtcataaaaccacaacggatagagagtaagacggtcaacgaatttatggatttagcactcttaggtatgaagtggcagcgtggagttactgaatatgctggatttcatacaatgaccatcgatgaatatatggactcactcacttttttaaagaagattgcatctttatttaaactggaaattcgatatcgtgttgagattaaaggttcaaaaatcatcggttggtatgtagatatgattcaaaaacgcgggcatgatacaggcaaagaaatagaattaggaaaagatttagtcggtgttacgcgtattgaacatacacgtaatatttgcactgctttagttggatttgtaaaaggtgaaggtgacaaggtaatcactattgaaagtattaataaaggtctaccctatatcgtagatgcagatgcatttcaaagatggaatgaacacggacaacataaattcggtttttatacaccagaaacagaagaattagacatgactccaaaacgtttactgacgcttatggaaatagaattgaaaaagcgtgtcaattcctcaatctcttatgaagtggaagcacaatcaattggtcgtattttcggcctagaacacgaattaattaacgaaggcgacacgatcaaaattaaagatacagggtttacaccagaattatatcttgaagcgcgagtaatagctggagatgaatcttttacagacccaacgcaagataaatatgaattcggagattatcgtgagattgttaatcaaaatgaggaattaagaaaaatttacaatcgtattcttagttcgcttggcaataaacaagaaatgatagaccagctagataaactagtgaacgaagctaacgaaaccgctagtaatgcaaagaaggagtcagaagcagcaaaagcactagctgaaaaagtacaagagaatattaaaaataataccgttgaaattatagaatccaagaatccaccgacaacaggactgaaaccttttaaaacgctttggcgtgatattagtaacggaaagcccggtattttaaaaatatggacaggtacagcgtgggaatcagttgtacccgatgttgaatctgtaaaaaaagaaacattagatcaggttaataaagatattgagtccacaaaaacagagttaaatcaaaaggttcaagaagcacagaaccaagcgactggtcaattcaatgaagtgaaagagagtttacaaggcgttagtcgtacgattacaaacgttgagaacaaacaaggtgaaatcgataagaaggtaactaagtttgaacaggattctagtggatttaaacagtccattgaagagttaacaaaaaaagacggtcagattactgaaaaagttaacactattgaatctactgtagaaggcacaaaaaagacaatttccgatgtacagcaaacaacaagtgatcttaagaaaacaacaactgaaattaaagaagaagctgggaaaatcagtgagaagttaatgagtgtagaaacaaaggttaatagtgataaagctggagggcgtaaccttttattaggttcaaatgttaaatatgaaaaaacagattacctaatcaatcaatattctctaactgaaaacttctttgcgggtgaggaatatacctttgtaattaagggaagcgtcccacaaggtcagaaatttgggatttggcagaatggcgggtctagcaatgttggatatgcaacaagtgtttatgctaatggaataacgtatgtaacctttaaagctgttgcaactacaagtggaaatgaaagaaagttaagtttatataactatccaagtaatactacaaaggcaattgtagaatgggttgccttgtataaagggaataagccacaggattggacgccagcgcctgaagaacaggtaacaaccgatgaattcaccaagaaaaccattgaaattacaaaaagtgtagatggtatcaaagaaacgataacaaaagtagaaaataatcaaagtggatttgataagcgtgttgctactgtagaaaaagatgcaactgccattaaacaaaatgtctctttaatacaaaatacgcagacagaacaaggaaggcaattacaagaggcaaaagctggatgggaaaatactgcaaaagcacttgaaggtaaagttgagcttaaacaagtagaggattatgttgcggggtttaagattccagagttgaagcaaacagttaatcagaataaacaagatttattagatgaattagccaataagcttgcaactgagcaatttaatcagaaaatgactctgattgataatcgttttactattaatgaacagggtatcaatgcagcagcaaaaaagacagaagtatatacgaaaacgcaagcagatggacaattcgctacaggttcttatgtaagagatatggaaactcgtcttcagttaactgaaaagggcgttagtatatctgtaaaagaaaatgatgtaatagcagccattaacatgagtaaagaaaacattaagttaaatgctgcacgaatagatttagttggtaaagttaatgcggagtggattaaagctggattgctgagcggttgccaaattagaacatcaaatacggataactatgttagtttagatgatcaatttatacgtctctatgaaagaggagttgctagagcatttctggggcattacagaagatcagatggtgcagtacaaccgactttcatcttaggttcagatgaaaagactaacgctccggaaggtactttgtttatgtctcaagcaggtgcaggatggtcaggggcttatgcgagcattggtattagcaatggcatagttgatggtgcagtccaaaagtctgtgtattgggagttgcaaagaaacggactaagtgttctaaacgctaatgattaccatgttttttacgctggaaatggaaattggtatttcagaagagggaaaccagggttgtatcaaacttcgttagtcgttgaagataatagtacagattctgatttaagattacctaatgtaactatacgtaatagccgtgcagcaggatatacaggagttattcaattgaaatcccctgttactcaaaatggatggggtgctgttcaagggaattttatgactccttcattacgggagtataaatctaatatccgtgatatttctttttccgccttagaaaaaattagaagtcttaaaattagacaatttaattataagaatgctgtaaacgaactataccggatgagagaagagaaaagtcccaatgatccaccattgacaacagaagatattaaaacatactacggtttaatcgtagatgaatgtgatgaaatgtttgtggatgaaagtgggaaaggaattcatttgtactcatacgcatccattggaattaaaggtttacaagaagttgatgcaacagtacaggaacaggaggtagaaatagcaaatctaaaatcacaaatagctagtcaagaagatcggatagcacgattagaagaattattactacaacaattaataaataagaaaccagagcagccataggctggtctttttattttggccaaaaaggagaggaaaagatggatcgtattgatgtattactaaaagcatttatagctgcgtttggtggcttctgtgggtatttcttgggaggatgggatgcaacattgaaaatcttagtgacaatggtagttattgattatttaactggcatgattgcagcagggtataacggagaattaaaaagcaaagttggtttcaaaggcatcgccaaaaaggtggtgctttttcttttggtcggagcggccgctcaactagactcggcacttggaagcaacagtgcaatccgtgaagcaacaattttcttcttcatgggtaatgaattactttcactcttagaaaatgccgggcgaatgggtattccactcccacaagcattaacaaatgcagttgagattttaggtggtaaacaaaaacaagaagagaaaaaaggagatgttcagtaatggaaatccaaaaaaaattagttgatccaagtaagtatggtacaaagtgtccgtatacaatgaagcctaaatatatcactgttcacaacacatataatgatgctccagctgaaaatgaagtgagttacatgattagtaacaataatgaggtgtcgtttcatattgcagtagatgacaagaaagcgattcaaggtattccgttggaacgtaatgcatgggcttgcggagacggcaatggttcggggaatcgtcaatccatttctgtagaaatctgttattcaaaatcaggaggagatagatactataaagctgaggataatgctgttgatgttgtacgacaacttatgtctatgtacaatattccgattgaaaatgttcgaactcatcaatcctggtcaggtaaatattgtccgcatagaatgttagctgagggaaggtggggagcattcattcagaaggttaagaatgggaatgtggcgactacttcaccaacaaaacaaaacatcatccaatcaggggctttctcaccgtatgaaacccctgatgttatgggagcattaacgtcacttaaaatgacagctgattttatcttacaatcggatggattaacttattttatttccaaaccgacttcagatgcacaactaaaagcaatgaaagaataccttgaccgtaaaggttggtggtatgaagttaaataaaacaaaagaatagttttatgaacaaaaataagagccgtcctgttgggcggcttttttttattgctcaattactgttgcactaattttaggcattcctgttttatctttttcgtcgtaggcgccatagattgttactattgatcctttagatatttttaatccgtttttaagtgttatttcattttcgttcgtttgcactccactttggacaatttgaatagtgtacatgcctttgccgtcattttcgtttgtgcttatgacaaatgaaggtaatgctgaagacttaagtaataaatctaccgttccggtagctttaagcctttttcctttttcgtattgatctccatttgccttaacaaaactaacttcttcagcatcttgctttatcttcttatttaactcatcctgagatgttaaatcttttttagtttctggttgagatttgacgttcgttttttcacttgattcactttgtttagaagaatcacaagctgttagacctaacaataaggtacttccaatgcaaatacttataagttttttatacattttcattctcctcctctatccaaatttcttccatgtgcaattttaattcctttgcaattttataggctgtaagaaaggtaggtagcgtcgtgttattaacgagtgaactcattgtagtttgactaattccaataagttttgaaaactccttttgacgtatttctctttcagcaaaaataacacgaagtttacattttaatcgcacaatatcacctctttaattatatacaattcgcatatggaaatgtgtcctcctttaatttaatcaacgaacatttagaaaagtttaaatggacaggcaatataactctttctaagtcatatacctatatcaagaccacgaggaataccaagtggaactaaggacatcaagaggggagaggattacatgcgttggcagtataatcacttgaatacaactccatatcttcatccatccaaagaattatgttcaatgtacaatggatcgagatcaagagcagagacggaatcaattttaaatcacatgaaaaatcatgaagtttatgatcgaaaagaatataaaggatatttcagtttgtcacaggtattagaagaagatctatatggagaggaagaagatgttttaaactgggaaattctaatggattgttatgatgtagttcttacaagaaaaggtattgcatttcgtgaaaaagaagaggaggaacaagcatgactcttgctggagaagcgattattatttggacggcaacagggttgtcagtagttgcaatgaaggcagcagaaaaaatggggaaaagtgttccacattggcttccacgtgtcactttgtacacaacacttacaggctcgtttctataccttctacgttatgttctcgttttatttctatgaaggaatacgatgtggaaacttttcattccttatgtcataaggagtttagcttgtatgcacgtattccttgaaacagggatatataccctctataagagggatataaggagtgattttatgctggagttgttatcagtaccattcgcaggtttaattttcgccatagttggcgaaaggctcaaaggaagagagagtgatcgaaagaaaatacaagttttttttgaagtaagcggaattgcgatacgtagagaggacaaattacagtatccagtttttcttgaacaaaaagaggatgaccgaagtacaacttatatatatcggttgcctgtaggaatgccgagtaaaattattcagaaggtcgaggatgttgtctctgaagggctaagtaaacctgtccgaattgattatgataattacaagctaaatattcgtgtgtttcatagggatataccgaaaaaatggtcatggtctaaaggtttggttgcagaaggaagctggtgtgttccaatgggccaaagtttagaaaaacttatctatcatgattttgataaaacaccacatatgacactaggtggtctgacacggatgggaaaaacggtatttttaaaaaatgtagttacttctcttactttagcacaaccagaacatattaatttatacattattgatttaaaagggggcttggagtttgggccgtataagaatttaaaacaggtagtttctattgctgaaaagcccgcagaagcttttatgatattaactaatatcctcaagaagatggaagagaaaatggaatatatgaaatgtagacattatacgaatgttgtagaaacaaatatcaaagagcgttacttcataatagtagacgaaggagccgaactttgcccagataaaagtatgaaaaaagaacagcaaaggttattaggagcgtgtcaacaaatgctctctcatatagcgcgcataggtggtgctttaggttttagattgattttttgtacacagtacccgacaggggatacattaccgcgccaagtaaaacaaaatagtgatgcgaaattaggctttagattaccgactcaaacagcatcaagtgttgttatagatgaagcgggattagaaacgataaaaagcattcccggacgcgcgattttcaaaaccgatagacttacagaaatacaagtgccttacattagtaatgagatgatgtgggagcatttaaaaggatatgaggtggagaaacatgaggatgcaaacgcatatgcaaatcaaccgtcaaatggcgatacttgcgacgattagaaagctacagtttgcaacgagaaggcatttaatgagtattcatgaaatgggtggaataagaaatgcaaatcgaattctgaaagatttatctatttatacaagtaaggtagtttacaataaagagcatgtatattatttaaaccaatcaggacataagttgtttggcgaagggaaagttgtacatcatggtaaagttacacacgctcttttacgtaatgaagcttggttaaatttatattgtcctgatgattggcaagtagaaactgaaattaaatatataaaggataataaaaagaaaaaaataattccagatgtgaaatttcgtgatgaggacagaatacttcatgctgtagaaatagatcgtactcagaaaatgatagtgaacgatgaaaaattaaaaaaatatgaggagttaacgcagatttataaacagaagcataacgggaaagtgccagttattcatttctttacaatcacaaaatatagagaaaagaaattagaagaactggcaaataaatataatgtgtttgtaaaagtatatgtaatcgctactacttaatgatgaaaaaaagagctgatcattttcgaatgattagctcttttttatgtattgtattacgtcgtctattttgtaaattttattaattcctttttctgcagcaatggcatttaaagcatcaatgatagcttcaagcgaatcaaaacgaacagcattagcattaccattcactaaatcactaatcgtgttgtatcttacttgggattctgtagataatttatttttagtgatccccaattcatctaaagaatttccgagtgtgaatttcattttattctcctccgcagcactggttatcttgtactcattttacaacatcaatcgaaattagtaaaacttttttcgttcaactattgacgttgaataattagagagttataattcaacttaaaaggaggaacaattatgaatcgagtaaatgattattttggtttagaaagtaaatcagattgcatttggttttatggtttcttcagtatatctacgattttatttttaatcgatatgattattgctcttatataaggaggggagaaaatgcttagctcagcaaactatacgcaatataaaaaattacaatcattccgatcagtagaagagatgaatgaagcgatttgttcttttttatacaaacatacacatgaattatccgaatcagcaataaaagtattgaaatttctagcaaggcactcttgtaaaatcccaggtgtctctttcttgaaggtagggacaattgcggaggcattaaatataagtgatcgaactgttcgcagggtactaaaagtattagaggattttgaagtagtaactagacataaaacaattcgaacggaaggaaaattacgtggagggaacggacataacgtctatgtccttctaaaaaaatatagtgtcacaccgaatgtcctaccgaaaatgtcacagcgacaagatgaagaaaaccttacagaatcaaaggtttcagatacaaaaacggacaaggaagctaaactttctgaatcacaccctctagaagaattgaaaagcgaattaaacgtaaaagaaacgtcagcaagggaatctaaagaaatcgaattagaggatctagatgaaacttttacaccagaaaatgtaccaagccaattcagagatgtggtagctccattcttcaaatcagcagataaaatttataaattgtatcatcgagtattaatagcttataaacgttcaaaaatagacaagcctattgaacaagtgataaatcaagccattcaagcattcaaagaaactgtcttcgcagaaaaagcaaataaaattagaagtacttttgaaggttatttttatagaattgttgaaagtaaatttgtaatggagagaaggaaagaatgtcgaggattattgttcgattggttaaatgaataatataaaattgcccacagggaaaaatatatatataatttaattatcatattcttagtaaataagtgggtgaaaattttgaaatacgctgtttatgtacgagtttcaacggatagagatgagcaagtttcatctgttgaaaatcagattgatatttgtcgatattggttagaaaaaaacggatatgagtgggatccaaatgcagtatattttgacgatggtatttctggtacagcttggttagaacgtcatgcgatgcaactaatattagaaaaagcaagacgaaatgaattggatacagtcgtatttaaatctatacaccgtttagcaagggatctaagggatgccttagaaattaaagaaattctaataggtcatgggatacgcttggttacaattgaagaaaattacgatagtttatatgaaggtggcaatgatattaaattcgaaatgtttgccatgtttgctgcacaattacctaaaactatatctgtatctgtttctgctgcaatgcaagctaaagcaagaagaggcgagtttattggaaaaccgggattaggatacgatgtaattgacaagaaacttgttatcaatgaaaaggaagctgaaattgtaagggaaatttttgatttatcctataaaggctatggatttaagaaaatagcgaatatcctaaacgataaaggcacatatacgaagtttggccagttatggtcgcatacaactgtagggaagattttaaagaaccagacgtataaagggaatttggtcttaaatagttataaaacagtaaaagtagatggaaagaagaaaagagtttacactccgaaagagagattaacaattatagaagaccattatccaacaattgtatcaaaagaattatggaatgcggtaaatagcgatagggcaagtaaaaagaaaacaaaacaagatacaagaaatgaatttagaggaatgatgttttgtaaacattgtggtgagccaattacagctaagtattcaggtagatacgcaaaaggaagtaaaaaagagtgggtatatatgaaatgcagtaattatattagattcaatcgctgcgttaactttgacccggctcattatgatgatataagagaggcgattatctatggattgaagcagcaagaaaaagaactagagatacatttcaatccaaaaatgcatcaaaaaagaaatgataaatctacagaaattaagaagcaaattaagttgttaaaagtgaaaaaagagaagttgattgatttatacgtagaaggattaatcgataaagaaatgttttcgaagcgggatcttaatttcgagaatgaaattaaagagcaagagttggcattacttaaattaacagatcagaataagagaaataaagaagagaaaaaaattaaagaagctttttcaatgctcgatgaagaaaaagatatgcatgaggtttttaaaactttaataaagaaaatcacacttagtaaggataagtatatcgacatcgaatatacattttctttatagttttaaagttggttattagttactgtgatactacctgcagtaacaccgatagcttgtccaagatcatgttgtgttaaattccgttctcttcgtaattgacgtaaccgatctttaaattccacaataatcacctcataagtggtttgttaggattattataatatttcctaaagggaaaatcaatccgagttatttctaagaataatataaaatatgtgtaaaaatatatcttgaattttccctaagggaatgttaaggtgatttacaaagatatagaaaggagttaccacatgaaagtaattaaagacgagacaaaattaaaagctgcattcaaaaaatctgggtataagtatcaagagttagctgacgaattagaaatatcctgcagctactgttacaagctaattaacaatcataattacaaaaagaaaatatcgtataacttagcatccagaatggcgcatgtattaaatgcaagtgtagttgatttgtttgaagagcaagtcgattttttttaataccaatattccctgagggaacataggggtgagagggccatgtcagaaatttattacaaagggtttatcatcaaggaaacttatggcgaaagaaatatcgaagaagtgtttaaagaagcatatgagtcattttatggggttgaagttaaggttgttaaaaaggaattagggactaaacgcaatagtgcagccagctaatctttaaacttcagtgagaacattcaatgaagtcgattataaaatggacaagcctgaaaggagagaaatgaatgaaaaacgggaaaaggttgactaaacgtgaaaaaatgcatcttaaatcatatagcttaaatcctgataattggttggttttcaagaaagcggatggagaaatgcatttagtacaccgttatactagcacaactcgtgtaattccaagtttataagtttaggagggaataagatggatcagttaacagtagcaagtgaattacgtcttttagggagaagaaaagtagctggatatgaatttactggaatcgagggaggatttggtgaaggtaaaaaagcaatgttggttttggatatagctacaattcataaccaaccattaaaagaaatcaatcgtcgcattaatgataatcgcattcgatttaaagatggtgtggatattgttgatttgaaaagtggtggctttaacccaccacaattattaaaccttggtttctcaaatatgcagatagcgaaatcaaataacatctaccttctatcagaacgaggttacgcaaaactattaaaaattctcgaagatgataaagcttgggaattatacgacatattagttgatgagtacttcaacatgagagaaaagaatcaagtggctacagatccaatgagtattttaaaacttacattcgaagcattagaaggccagcagcaagcaatcgaagagataaagtcggatgtacaagacttgagagaaaatacaccattatttgcaattgaatgtgatgaaatctctacagctgtaaaacgtcaaggagtcatattgttaggtggaaaacagtctaatgcctatcgaaatcgtggattaagagggaaagtttatcgtgatatctacaaccaactataccgtgaattcggagtgaaaagtcacaaagcaattaaacgttgtcacttaaatgtagcagtaaaaatagttgaagaatatacacttccaattgtattgagcgaagagatttcttttgtaaatgcacaaatggattttacagaaatgtagttagttaaaacattctcaaccggtttttttctaagttaaaaatttaaagaaaaggtggaaaagacaatggaccagttacgtgttattgagggagaaaaagtggataagccagattatgttgagatataccttggagcatttatgaatgcagttaatgagttaaagaaacaggatgaggaaacgagatcattaagcaaggatacgtataaaaaagcaattttttatggagttagatacatttcaatatcaaaaaatgacagtttgaattatgactacctaatgaatagatttcttttaataagctatttagaaaatttgatgaaggtgttgacgcctagggattttatgaccatattcccaatcgataaaaattatgatggcgctcgttatgaaatgaaagattacttttttaccatgaatgaaattaaaaaaatcggaatggatacacctattggagagaaaatcatggagtttttatgggattaccaaaactttaaagatataacactatttaacttagcctctgtaagcattttaaataaattgcagaaaatgcaaggtaaaaaaacgttaactgaagagtttgccgagcgattaggtatcgatacttacacgaagcataaagaaaagggtggaaaagaatatattacaaatgaccgtactggtgagatccaagaagttaaaaaatctagaccaagatatttaaaaccagttcaatgattgatgttattaaggcttataaacaaagaaagtaacttgcgccaacaagttactaaataaaaatacttataaaaatatacttattagaaatataacatacacactcgatgtatggaaagggtgttattatggctctttttagaaaagtgcatacagaattttggacagacgtaaaagtatcagaagatatgacgccagaagacaaattgtttatggtgtaccttttaactaatccccatacaactcaattgggagtatatgaaatcacacctaagatgatagcttttgaaatcggactatcaatagagtcggctagagcactattggaacgttttgaaaaccatcataaattaattaaatataacaaactgacaagagaaattgctataaaaaattggggcaaatacaacctgaatagaggcgggaaaccaattgaagattgtcttaaaagagaaattgataaagtgaaagatttatctctaataaaattcattttagaacatacagatcatgcagctttaaaaagaaaaatcaatctttatgcgggttttgacgatacgtcccacgatacgttagcgatacgtgaccaagaagaagaaaaagaacaaaaaaaagaacaaaaagaagaacaagaagaaaaagaaaaagaaaaagaaaaacaaaaagaagaagaaaaagaaccagaagaagaaaaaacaagaataaaatccaaagcgtctttaaaatcagacgcaaagtccaatccaataccgtataaagatatattggattacttgaatgaaaaagcaaataaaaatttcaatcctaaagcagaaggacatagaaagttaattcgcgctagatggaatgaggggtataaactagaggactttaaaaaagttatcgataacaaaactacgcaatggtttggtaagaaaagttttgatggaaaaccactagatcaatttttaagaccgagcacgttatttgcacaaaaacattttgacaactacttaaatgaaacggtcaacatatccaatcaacaacatggagatcagattgttatacctggatttaggggggaaatgccgttttagaaaggagtactaaatgtgaaaaagatacaagattcttttgaaaaacttactaagttaaaatttgcagatgaacaatgtgataagcacacctttaataaacatgggaaagaagttattaaattagttaggaaaatgattgatgatgcaggaacggtatattgtccccgctgcatggttgaagagcaaaattcagttttatttcaacaagcaaataatcattataaaaagattaatagagaacggaagaaaaatgtactctttcaacacagcatcatagaaaatcaatccattacagaatcaagattgtctacatacaagacggattgtcaagaaacgaaagaaaacaaagaaaaagctataaaaattcttgaacgcataaaaaacggtgagtttttaaatgtatacattgcagggattcaaggagtaggaaaaagccatttagcgtatgcgatgctgtatgaattagttaaacactattgggtaatatcagacggtgagaaattaaatgacgaacatgcttttaaaaatatgaaaagctgcttatttgtagagattgaaaagctaattcgattaatacagcactcttttagaaatatagagtcaaaatatacaatggattattgtatcagtttaatggtagatgtggatttccttgtaatcgatgatttaggagctgaaagtggttcgatgaatcgaaacggagaagcaagcgattttgttcataaaatactttatggtgttacaaatggacggcaaggagcaaataaaacaacaattacaacttcaaatctgtcaagcgctcaattatttcaaaaatacgatccgaaactagcaagtagattgttaaacggtgtatcgaaagatgaaacaattgtttttaaaacaaccactgacaaacgaattgtaaatttagacattggattctaataaaaggggtgcggagaaatgaaagaggtaaaggggaaaaacaccaaattaatggaagaatttgacgtgttattaagacaactgctgattaaatctaaaacagatgaaagggtaaaaaactttttggatgatctgtttgaaatgctaagtgataataagctgcagtctgatattgatttcaaaacagcattaaataagttaagagaaaagcactttcctaagtttgataaaggagagagcaaaaatgactaaagaaaagggacaagctaaggaagtagttaatgttcgtggaatgtcagatgatgagtttatagagaaatacggaaggcttgtacatcattgcgtatggaaaagatatgcgaaaaaaaaggccagtatagagcgtgataccggtttagatattgaggatttaacacaattcggaatgatcggtttgataaaggcgcgagataattttgaccttgaatttggatgtgcgttttcaacgtatgctgttccgaaaattattggggaaataggaagggcaattcgggataaccaaaaaataaaagttcaaagaaccgtatatggcgtaaaaggaaagattttaaatcaacagttagcagataaagaaccagaagaaatagcagacattttggatgagtcagtatctttagtaaagacggctttagagtatcaaccaagcacagattcactcaataaggttgtatatgcatctggagctaatgaagaactgacattagaaagaatgatagaggatactaaaacggaagacattgaagaaacaaccattaatcgagctgtgataagagaatttaaagctgcattgcctcctaaagaatatatcgttttagatatgcgtttacaaaatatgacgcaacaaaacattgcaaatcaaatgggatacagtcaggtacaaattagccgtatattagcaaagattaatcaaagagctgctcaatttggtaaagaaggagggcttcaagattgagtgttacaaaaggtgtttgtatcgatgtagatcactcagatttgctacatgagaaagtagagtactttttattccctgctaaaccaagtcattactatgtaagcagatttaatcgtaaaggagcgcattttggttgttatcaagctgaaaggtttcaaatcacggaaaaggaagtatggacaccagaacctcaaccgaatctgcctgagttgaatacaagcttattctatagagctcagttgatttggcgaaaaaaggggtataaagataaaccacttaaagactacatcgtacagccgagagggaaacattgctacttttggcatgatcgggagcgaaagaaattttgtggctgttttccgctacattggtttaccgattttgtaccagttcaaagtcatcatatagaagaaaaaactagagaagaggttaagttattacaacggccagatggacaacttgcatttttttaacgaaagaaagtgaatgggcgttttacccagtcatcgatttaaaaaaaggagtgttcgtaatggatattaaaaagttatttgcaatgcagaacattttggataaaagagttttagagtcaaaaaatctttctagaggagaagtattcgaatttagaatactagcgtttttagatgaattaggcgaatgcatgaaggaatggcgagtatttaagttttggagcgacgatcgtaaaccgagaactagcatacctacaggggaaatcatagtactagatgatggttatgaagtagaagtttataaaaaccctttacttgaggaatatgtggacggactacattttgcaattggactttgcatagatttgaaaacagaaattaactttcctgcttctatgcgttgcgagacagttacagagcaatttttcgaattgtatcatctagcaatacgattaaaagaagaaccgacagcatttagggcagatgttcttttatcccattatcttggtttaggggaattgttgtgcttttcgttagaagaaattggacatgagtacattgagaaaaacaaaatcaatcatgaacgtcaaagtaatggatactaatacaatttgaattttgttaagaaatgagggtgattgaaataagttggtgggcaatagcgatcggtttatatctattgattggagttgcattacttatatggataatcgcaacggatagttggggttcgttattcttatatcctgtttttgcggtagtcattgttttgggatggcttccattaatgataagaagcattgtacaagagatatctaaagcgattcataagtggaaaagaaagcagaaaactgaatagaagtattatttcagggagggagaataaatgatttatgaagttacagattattgcagtcagtgtgatagaaaaatagagaattgcgattgctgttgtaataagtgtgatgagtggttgcacgattgtaaatgtaaagataaataagcaaaaaaggggaatgaaagatatgaaatggatgtacaaccttgatagcaataatgagatttggacaagcgataaatttgaaatgaaagaagaagctattcaagcagctttaaaagattggacagataaaatggtagcggatagagcggcagtcgataatgaattccaaattggacaattcaaacagtattctccatggatcaatgcagatgtattgttggatgaattgtatgaacgagcaaccgatgaatgtggagaggttgcggaatattggctttcaggtgtgccgatggacgaaggggaaaagcttcaagaacaaattaataaggtagttacagaatggctaaaaggaataaatgagcatcctagctttggttcaattgaaaatattgaaacgatagatgctagcaaaattgaatataaagaaaactaaacaaaagcgttatttgataaaaaataagaaagccctagctttcttattatatgtaaaaagtcatatgttttttatcttctttatagtactctaaccggttttgcaaagtgccagtgtggaactcaaacttatggccatctggatctgtaaagtaaagagatctttggtctctctcatctctttctcggccaggtaaaatattaacatcattttgaattaatacttcttttaaatggtctaatgcttcattagttacagtgaaagccatatgtgtataagattgcttaatttcatttcttggtatatcttcttcaacatttaaagcaatccataatccatttaaatcaaaatacgctaattttctaccttttactaataattttgcttgaagtatttttttgatagaattcaatagatttttccaagtttgatacagaaaaacaaatatggttaatgccctgtagcataaaaaacgccccctataattaaatgattttcaatatttttatataaagattataaaagtttatgcgcgatttataaaggattactacaaaatagttatttgaattaaaaagagcgccgttggagagtgcggtgctcttagaccaagaactataacagggattaaggaaagaatattgtataccaaattgatagtaatgcaagccatccaattgtcagcgctatgtattttaaaattttcatgattactccttttaggtatagagtgcaccaagcaagaggatgttattaatttttaaacaaaatgcttatttaaaaactaaagagggctttttaaagcgctccttaagaaaaataaaaaagaatacctcatgatactgtatgtatgtttttttaggaatgtgaggatttaaaacaaaatcgttattttatagatcggagtgaaattcaaatgattgttaaagcgacaataaaacttgaattagatgattcgcagaaaaattgggtttcttatgttagagaacaaggtggagaagaagcggtatttcattatctggaagaagaagtgcagaagaaaattgaattagctgattttgtggagatgaaatacaaaaataagtaatttaaaccaaaacgctattttataaaataaaacagctagcgtgattagctagctgtcctgttaagaaaagaaaacggtgtttagcaaatgttgctgttgtaattgcgaattacaaccatagtatgagcagaagtaaaaatgttatgcaagaaagttaaataaaaactgcattttattgaaaagggggaatggatatgtctctagtagggaatttaaaggaactccaagaaaaagccatcgatgaaaaggtattggaatttgcggaagaaatggaaatcgtaataactaaaagtgccgcaagcggatattcaggtcatagatataagattcataatgaaaatccaaatcggcatatgatgtgttcaaaaatatttatagaaaagttacaagaattactggacggtgtgaaggttgaatttaaggaagaagaaaagaaaaatattttaggcggatcttactacgaacattacatccgttttaagtggaatgactaatttcttattaaaaattttattttggagaaagggagtagaaagaatgaaaacttttaatgtgacttttacagagttgaaaatatatgaagcagtcattgaagcggagtcagcggaaaagattattgatgtgattaaacacttaaaaagaactgaagatgatttagtagacaaaggagtcatcataaacgaagttagtgagataaatgttagtaaagaacaaaagttcgaataaatcaacttctcagattgtttattttgagacggaaacaactttctgaatatcataagaccttattagcgaaaaaactcttattcgagcgtacaagcctgttatacacgttgcacggaaattagaatgaatttgttaaggaaggaagtataaaaatgagggcttggaagaaaaaacatgttaaaagagcatttttgaatcgtcaaaaggaaattgataaagaacggactgctgcagcttggagaaatatttttgtgaaatcaggaatcataaaataaaaaaggaaaagcaactcgttggggacaagtcacttttccagatggcaatgtaaatccattatagcaaaacatatgtacaagctgtagcaataaacaacgagatattttgacacctatcgacaattagaaatgtggttgttgatctagaaatatgaaagtaggtgaatcatcatttgtttaactggctgagagattaccaaaagttagaagaagacatagcctatctggaatacaacttagataagacaaaagctgaattaagacgctgggtgagtggtgatttgagagaagtacgtttaacggcagaatctgaaggtgcaaaagttgaaaaccgcattgaagcgattgaatacgaattagcacataagatgaacgatatgtataaattaaaaaagttaattagtaagtttagaggtttagaaaatcagatactcaaattaaaatatgtggatggtatgacgttagaagaaatagcagaggcagtaaattatagttctagtcatatcaaaaagaaacatgctgaactcgttagattaattaagttcgtggagcgagaaggtgtcatttaggttcactcctaaaatgaatcgaaacggttgaaaaaatgatttatattgatagcatacaattttagcagaagggcaactggtgcacggttgctctttttgattttggaggttattagacgatggatgtacaagagttgtcgagacgattagaaaatctagaacataaagtgcttcaggtagaaacgaaggcagatgtgctaaaccgaacagctatacaaaaaggcgataaaataaaagtggtgtatccgcatttagggatacaaggcgagtatttagtggagaaaattgataatggtgtgttggaattggtagcagaagaaacaatgaaaaaaatacaggagtgattaggattgaagaagttatctaaacaagagctagcagctgtaatgacacattgtatttcaacgcttggtgagcagattgttaatgagcatattaatccccagaagttggcgcaagcaagtgcactccataacgatctctttgataataccactcctaaagaacgtagggaagcgacgatcagtttactagggaaagcgattgatgagtttttagagagtaaggagtgaggatatgggaaagggatattttaataaggctgtatgtttagtgtgtggtcatcaagatagagtgaatcatccatctaaaaaagagtatcaagaagtaacggtttgtccggaatgcaacggtgcttttgtagatgtgtggaagctaggaaagtacaaacgtaatacacagtctaatgaagaacctttattaacaattacattaacagatatagatgctaaaccgatagttcattacaaaggtgaacagatagatagaaagttacgtgttacgtttgattgggaatctcaatcgattgataaaattaatcggacatacattcatattgaacatgtaccagccgataacaaacgtttaaataccgagaccattcagcataatcatcctattgcaaataaggaacaagtttagatgttgtccatatttgttaataggtaaaagataagtgttttatctggaagttcaaacgtgaattaaagaaattaaaaaaggaatatgaaaaggagagtcactgaatgaacgggtttaataaaattgtaaacgatatgcaaaatgaacaagtaggaaatgctatgctagattttgctttggccgctaaaatgatgttcgctgcctttacacagtttaaagaagctggatttaacgaagagcagtcattcgaattaacacgtgagatattaattgattcattaagtaagaatcaatagatcaatgaggtgaaagggaatgcaagtatattgctctgagtgtgataaaagttatgacatgcagccgcaagtaacacaactccctaatcgtattgagaagtgtttctttatttgtcctcattgtaatcatgaacatatagctgcgtacgtgaatgataagattcgtaagtatcaagcagatatagcaaagtgtcatgagcggattaataaaaagaatcttgctatcgaagatgaaatgaaacgattaaggaagaggtttgacaggagaaagtgagaggtgaagcgagtttgaaaatgctattaacaaagcattggtgtttagatagaaactgcggatttgaagagacttctcataaggtacgtgatggttggaaatgtcctgattgtaatggaccaatggcgtttcaacaggtgaataagaaaaaagaaagcgccaagtgatggtgctttttattttggaggaggatgaaggatggaaggacaggagttaacattggaaaagaaagacagtatttatcttagaccaagataccctcataagattgacgcaagtaaaatcaaatccttaaaagatgtaattaagattttaggattgatggatattcgtttggacgacaaggcggtcattggtctagaacacttgattgaaaaggaggaagaataaaatggccaataacaaattaattattgaagtaactgcggatacaactgaggcattagaaggaattaaagaagtaactgaagcagctaatgaatgtgcagatgcgctggacaaattagaaaagattatggataagtttacaaatcgaagtgatacagtggaactctattgtgaaggtaaattgttatcgaagtctacagttaatcatacagctgattcaattcaatgtcgcataatcaagggagaagagcttggaggaagtgaacgctgatgaagaaaccgcttagaccatgctgcgaatttcattgttataatctcacacgtgaaagatattgtgaggaacatagatacaaagagaaggaaacgcagcaggataagaatagatactacgaccgattcaaacgggacaaagagagtacggctttctataggtcaaaggcatgggaaaggttaagagagcaggcactaatgagagacaaagggttgtgcctacattgtaagaacaatagaaagattaaagttgcagatatggttgaccatatcattccaatcaaagttgatccaagtttaaaactcaaattagaaaatttacaatcactttgtaatccatgtcacaacagaaaaacagcagaagacaaaaagaaatacgggtaggggcgggtcgaaaaacattcagggcggtctgtccgtaccgccgcccc